(19) United States
(12) Patent Application Publication
(10) Pub. No.: US 2024/0225047 A9
(43) Pub. Date: Jul. 11, 2024

(54) FERMENTED PLANT PROTEINS

(71) Applicant: DMK Deutsches Milchkontor GmbH, Zeven (DE)

(72) Inventors: Christian STADLER, Zeven (DE); Jan POMMER, Edewecht (DE); Chris SIEDENTOPP, Zeven (DE); Arthur SAGURSKI, Edewecht (DE)

(21) Appl. No.: 18/375,636

(22) Filed: Oct. 2, 2023

(30) Foreign Application Priority Data
Oct. 21, 2022 (EP) 22 202 893.8

(51) Int. Cl. A23J 3/34; A23C 11/10; A23C 20/02; A23J 3/14; C12P 21/00

(52) U.S. Cl. CPC A23J 3/346; A23C 11/103; A23C 11/106; A23C 20/02; A23C 20/025; A23J 3/14; C12P 21/00

(57) ABSTRACT

Proposed are fermented plant proteins obtainable or obtained by the following steps
(a) providing a plant protein source;
(b) admixing the proteins from step (a) with water to obtain an aqueous protein phase;
(c) admixing the aqueous protein phase from step (b) with microorganisms suitable for fermentation of proteins and/or with proteases; and
(d) fermenting the aqueous protein phase from step (c) and optionally
(e) inactivating and removing the microorganisms and/or enzymes
and their use in vegan dairy substitutes.

### FIELD OF THE INVENTION

The present invention is in the field of vegan foods and relates to fermented plant proteins, to a method for producing them and to their use for producing vegan dairy substitutes.

### TECHNOLOGICAL BACKGROUND

Vegan foods are understood to mean products intended for human nutrition that are solely plant-based, examples including vegetables, fruit in the culinary sense, fruits in the botanical sense and nuts and also preparations produced therefrom. The trend towards a vegan diet has increased greatly in the last 10 years. According to the German Federal Ministry of Food and Agriculture (Bundesministerium für Ernährung und Landwirtschaft, BMEL), 10% of the German population were vegetarian and 2% vegan in 2021—twice as many as in the previous year for both groups! Since 2017, the launch of vegan foods and beverages in Germany has had a share of 15%.

The trend towards vegan alternatives is therefore also not passing the dairy processing industry. According to a publication by Future Grocery Shopping, up to 93% of consumers in Germany are now regularly purchasing plant-based dairy alternatives. Sales have increased by 42% between 2018 and 2020. This is mainly due to the growing popularity of oat milk. In spite of the rapidly developing market, dairy substitutes, such as vegan milk or vegan cheese, are often far removed from the original in terms of their sensory characteristics—taste, texture and colour. For example, mention should be made of the typical bitter taste of many plant proteins required to produce the substitutes, and the multitude of auxiliary substances and additives which are required to imitate a corresponding texture and which should be reduced in amount PRIOR ART

WO 2020 089383 A1 provides a vegan cheese analogue which is produced on the basis of dietary fibres, calcium, lipids and plant protein.

Document EP 3213638 A1 discloses a cheese analogue which comprises water, a root or tuber starch, native potato protein and a fat component.

### Object of the Invention

It is therefore an object of the present invention to remedy the deficiencies of plant proteins with respect to taste, texture and colour and to provide vegan protein-containing dairy substitutes having accordingly improved sensory properties.

### DESCRIPTION OF THE INVENTION

In a first aspect, the invention provides fermented plant proteins obtainable or obtained by the following steps
(a) providing a plant protein source;
(b) admixing the proteins from step (a) with water to obtain an aqueous protein phase;
(c) admixing the aqueous protein phase from step (b) with microorganisms and/or enzymes suitable for fermentation of proteins; and
(d) fermenting the aqueous protein phase from step (c) and optionally (e) inactivating and removing the microorganisms and/or enzymes.

In a further aspect, the invention provides a corresponding method for producing fermented plant proteins, comprising or consisting of the following steps:
(a) providing a plant protein source;
(b) admixing the proteins from step (a) with water to obtain an aqueous protein phase;
(c) admixing the aqueous protein phase from step (b) with microorganisms and/or enzymes suitable for fermentation of proteins; and
(d) fermenting the aqueous protein phase from step (c) and optionally
(e) inactivating and removing the microorganisms and/or enzymes.

It has been found that, surprisingly, fermented plant proteins have distinctly improved properties and can be used to manufacture vegan dairy substitutes that are hardly inferior to milk-based products in taste, texture and colour.

The following changes in the properties of plant proteins are observed in the fermentation in accordance with the present invention:
Colour: Brightening of colour is observed, especially after pretreatment of the proteins (e.g. heating, hot-holding or high-pressure treatment);
Taste: Certain flavours are formed, undesirable sensory properties such as the typical bitter taste of plant proteins are neutralized, and species-typical taste such as “a bean taste, a pea taste, an earthy taste, a mushroom taste” is reduced. This can avoid or reduce the use of flavour enhancers, for example in the form of yeast extracts. Furthermore, an acidic taste note is formed that resembles the sensory profile of conventional dairy products, and cannot be accentuated in comparison with an acidifier such as citric acid;
Odour: A natural acidic note is accentuated in the odour of the end product as a result of the fermentation and it reduces the need for flavourings and acidifiers and thus comes closer to a conventional dairy product;
Consistency: As a result of the fermentation, the vegan dairy substitutes have a creamy consistency that is difficult to achieve with the use of additional additives or conventional process technology. A longer mouthfeel is thus also produced. Undesirable sensory properties such as: “sticky, floury, suety, gritty, greasy, short, brittle and firm” are reduced or avoided altogether;
Quality: The production process influences the viscosity, which proves advantageous when processing and also ultimately leads to an improved function in the end product. Water and oil binding is improved, thereby making it possible to reduce the amount of emulsifiers required in the end product.
Properties in the end product: Structure formation during application in the form of elasticity, pull elasticity, water binding, oil binding, gas/hole formation, foamability (stabilization of air bubbles). Taste formation: metabolism produces flavourings, thereby making it possible to reduce flavour enhancers and/or further use of flavourings.

### Plant Proteins

Plant proteins in the context of the invention can be obtained from broad beans, oats, rice, almonds, acorns, potatoes, soya, peas, lupins, rapeseed or other high-protein fruits such as sunflower or pumpkin seeds.

The harvested protein-containing fruits are mechanically comminuted and defatted. Flakes or a high-protein powder are the result. Thereafter, solvents are used to obtain a protein concentrate, which may be further purified and concentrated to form a protein isolate: The flakes or flour are admixed with water and mashed. In the next step, the low-protein fibres and solids are separated from the high-protein solution with the aid of industrial centrifuges. This is followed by so-called precipitation. Here, the pH of the high-protein solution is adjusted to the isoelectric point. This causes sedimentation of the protein particles. They are then in turn separated from the liquor by centrifugation. In order to remove all the constituents of the mother liquor from the precipitated and separated protein, the protein is admixed with water once more and separated again with the aid of centrifugal force. In the case of dry extrusion, heat, pressure and auxiliary substances are supplied to produce an intermediate product having a low water content. These likewise suitable proteins are referred to as TVP (Texturized Vegetable Protein) and have a dry consistency in the form of grains, strips or flakes. In the case of wet extrusion, the alternative is to work with a higher water content. The moisture content of the intermediate product is therefore closer to the water content of the end product. The intermediate product is referred to as HMMA (High Moistured Meat Analogues).

All starting variations of the proteins can be used, namely raw state and shelled/ground, raw state and shelled/ground/air-separated, raw state and shelled/ground/concentrated or as protein isolate.

It has been found to be particularly advantageous to expose the proteins to, and thus pretreat them with, increased pressure in the range from about 20 bar to about 250 bar and/or increased temperature from about 75° C. to about 95° C. with a hot-holding time of 5-10 min. Since proteins are thereby optimally dispersed, sedimentation during the fermentation is thus reduced and bacteriological risks are substantially minimized.

Later in the method, the optionally pretreated proteins are dissolved or dispersed in water which also comprises, as desired, salts such as sodium chloride, sodium citrate, calcium citrate, sodium phosphate and calcium phosphate, preference being given to setting a dry mass of about 3% to about 25% by weight and, in particular, of about 3% to about 20% by weight. This ensures that the proteins are stabilized during heating and ensures some of the salt concentration during further processing.

### Plant Fats and Dietary Fibres

In a preferred embodiment, at least one plant fat and/or at least one dietary fibre can be added to the aqueous protein phase from step (b), wherein the preferred amounts are in each case about 10% to about 25% by weight and, in particular, about 2% to about 10% by weight. However, as desired, the fermentation may also be carried out without the use of plant fats or dietary fibres. In addition to the changes as regards taste, colour, performance and influence on consistency, the use of plant fats is advantageous because it means burn marks and biofouling in the heating systems are less severe.

Suitable plant fats are selected from the group formed by cocoa butter, coconut oil, palm oil, palm kernel oil, solid coconut oil, nut oil, shea butter, apricot oil, soya lecithin, sunflower oil, soya oil, rapeseed oil, nut oil or mixtures thereof. As a result of the addition of the fats, the proteins are stabilized throughout the process against thermal stress and decomposition. The fats can therefore also be added as early as at the pressure/temperature pretreatment stage. They additionally have the function of binding the flavourings produced during the fermentation.

Suitable dietary fibres are plant fibres, for example selected from the group formed by citrus fibres, coconut fibres, spelt fibres, oat fibres and apple fibres. They have revealed structuring, sensory and performance advantages during the fermentation, since these starting materials involve an input of dietary fibres and carbohydrates. The vegan dairy substitutes subsequently have a more full-bodied taste and require smaller amounts of emulsifiers, of stabilizers and of substrates required for metabolic activity.

### Microorganisms

Microorganisms suitable for fermenting proteins are, for example, selected from the following group: mesophilic and thermophilic microorganisms such as those originating from the genera Streptococcus (e.g. S. thermophilus, etc.), Lactococcus (e.g. L lactis, L cremoris, L diacetylactis, etc.), Lactobacillus (e.g. L. acidophilus, L bulgaricus, etc.), Leuconostoc (e.g. L cremoris, etc.), Bifidobacterium (e.g. B. bifidum) and combinations thereof.

### Enzymes

Suitable proteolytic enzymes, which are also referred to as proteases or peptidases, include aspartyl proteases (e.g. pepsin, chymosin, cathepsin E), cysteine protease (e.g. papain, cathepsin K, caspase, calpain), serine proteases (e.g. chymotrypsin, plasmin, thrombin, trypsin, granzymes, kallikrein) and threonyl proteases (e.g. proteasome) and mixtures thereof. The individual proteases have specific pH ranges in which they each have an optimum effect; said pH ranges are well known to a person skilled in the art.

Optimal enzyme activity is ensured and the best results are achieved by varying a hydrolysis time of 10-120 min depending on the dosing amount (doses of 0.01% to 0.5% have been found in ideal circumstances) and hydrolysis temperature (optimum 45-55° C.).
In a further embodiment, the addition of enzymes in vegan dairy alternatives in which plant proteins were used for production has been found to be advantageous. In addition to time and process advantages during production, there are improvements in consistency, mouthfeel, colour and taste.

### Carrying Out the Method

The proteins are degraded by admixing the aqueous phase with the microorganisms and/or enzymes. Proteolysis is preferably carried out in a stirring tank having a continuous inflow and outflow and a dosing device for addition of the microorganisms or the enzymes and a valve located at the bottom of the reactor for discharge of deactivated microorganisms/enzymes which sediment over time.

It has been found to be advantageous to set, for example, an effective dose of microorganisms in the range from about 0.01% to about 0.1%.

If enzymes are used for the fermentation, the concentration should preferably be about 180 000 to 250 000 FCC units of protease per kg of protein. The dosing amount can be used to vary the activity and fermentation time in combination with the temperature. For example, the fermentation time can be shortened at a higher dosing amount. Doses of 0.01% to 0.5% had been found in ideal circumstances.

Temperature and pH depend on the optimum of the particular microorganisms or enzymes used and can therefore vary greatly. Generally, however, the fermentation is carried out at a temperature in the range from about 20° C. to about 55° C. and/or a pH in the range from about 4.5 to about 7.0 and over a period of 5 to 24 hours. If necessary, the microorganisms or enzymes can be inactivated by heating at 70-95° C. for 4 to 600 sec. and/or shifting the pH.

The fermentation can be followed by a purification and/or concentration step. Furthermore, it is possible to carry out the fermentation in multiple stages, in particular in two stages, for example with microorganisms in the first stage and with enzymes in the second stage or vice versa. The consecutive use of different microorganisms or enzymes is possible, too. Purification and/or concentration steps can in turn be inserted between the fermentation stages. Thereafter, the fermentation product thus obtained can be added to the vegan dairy substitutes in amounts of, for example, about 5% to about 20% by weight and preferably about 10% to about 15% by weight.

In a further alternative embodiment of the method, the microorganisms or enzymes can be added during the production or just before the filling of the vegan dairy substitutes. As desired, functionality or activity may be stopped by a further process step in the form of temperature treatment, pressure treatment or the like before the filling of the end product or in the final packaging of the end product.

### INDUSTRIAL APPLICABILITY

In a further aspect, the invention provides for the use of the fermented plant proteins or the above-described process product for production of vegan dairy substitutes, to which they can be added in amounts of, for example, about 5% to about 20% by weight and preferably about 10% to about 15% by weight. The term vegan dairy substitutes is to be understood to mean vegan foods which imitate dairy products with respect to taste, texture and colour and which contain plant components only, such as plants fats and plant fibres. Examples include vegan milk (oat milk, pea milk, soya milk), vegan cheese, vegan yogurt and vegan desserts.

Also claimed are vegan dairy substitutes containing the fermented plant proteins or the process product and a method for producing them, comprising the following steps:
(i) providing a vegan dairy substitute base
(ii) providing the fermented plant proteins or the corresponding process product; and
(iii) mixing components (i) and (ii).

### EXAMPLES

### Example 1

### Production of Fermented Pea Proteins

9.5 kg of water were heated to 55-60° C. Thereafter, 10 kg of plant fat adjusted to a temperature of 60-65° C. and 1.2 kg of pea proteins were dispersed at a speed of 1800 rpm by means of a dispersing system. A dry mass of 7% by weight was set as a result. Thereafter, the pea protein/plant fat/water mixture was heated to 95° C., hot-held for 5-10 min and cooled to 40-45° C. Once the cooling temperature was reached, 10 g of thermophilic acidification culture were added to the mixture.

A pH of 4.5 was reached after 24 h at 40-45° C. owing to the microorganisms of the acidification culture. The beige mixture coagulated due to the fermentation was then agitated and was used for further processing in vegan dairy alternatives. Acidification activity can be quickened and, depending on the objective, further lowered by using a substrate in the form of, for example, dextrose. This ferment with active microorganisms can thus be used for further processing in dairy alternatives in order to influence, though the active culture, further functions such as consistency, colouring and flavour formation or elimination of negative protein flavours. Also conceivable before further processing would be inactivation of the microorganisms by heating to 85° C. and hot-holding for 5 min.

### Example 2

### Production of Fermented Broad Bean Proteins

In a further possibility, 28.5 kg of water were heated to 55° C. Salts such as sodium chloride, etc., or a substrate such as dextrose can also be added as desired to the water and can be dispersed by means of inline dispersion. Thereafter, a 30 kg plant fat mixture, likewise heated to 50-60° C., and 3.6 kg of broad bean protein were premixed by means of inline dispersion. A dry mass of 7% by weight is thus set. Thereafter, this mixture is heated to 70° C. and homogenized in two stages of 200/50 bar by means of a high-pressure homogenizer. After homogenization, indirect heating to 127° C. is carried out with hot-holding for 4 seconds and subsequent cooling to 40° C.

Afterwards, this pretreated mixture is inoculated with 0.05% mesophilic culture and fermented for 24 h at 40° C. up to a pH of 4.5. Once the pH is reached, the ferment is agitated and provided for further processing. Here, concentration may be carried out by ultrafiltration, which would make the concentrate brighter in colour. The concentrated ferment would be provided for further processing to form vegan dairy alternatives.

### Example 3

### Production of Fermented Vegan Cheese Alternative Containing Plant Proteins

For the fermentation of a vegan cheese alternative, all the ingredients such as salts, stabilizers, starch, emulsifiers, plant fats, plant proteins, colourings and flavourings are premixed by means of a dispersion system, pasteurized and cooled to 40-45° C. Afterwards, the culture solution, already prepared in a pasteurized 30° C. saline solution (e.g. 0.09%), is aseptically mixed into the cheese mass. Thereafter, filling is carried out under aseptic conditions into shaping bags. After a fermentation time of 24 h at 30-40° C., the pH is lowered to 4.5. Afterwards, cooling is carried out to 6° C. and after another 24-48 h, the vegan cheese alternative is processed in such a way that it has the properties of a feta.

### Example 4

### Production of Fermented Yogurt Alternative Using Enzymes

3.5 kg of pea protein are dispersed in 56.5 kg of water. This is followed by first heating the mixture to 85° C. before cooling it to 55° C. and transferring it to fermentation tanks. In the next step, proteases are added at a dose of 0.01% to 0.5% based on the protein content. Hydrolysis is carried out over a period of 10-120 minutes with the stirrer switched on. Subsequently, the product is heated to 95° C. for 10 minutes in order to inactivate the enzymes. Finally, cooling is carried out to 8° C.

For further processing, the dry substances are added to the enzyme-treated starting base. Thereafter, the mass is heated before the preferred fat is added. Later, the product is preheated before a two-stage homogenization is carried out. Subsequently, the mass is heated to 95° C. and then cooled to 41° C. In the next step, the cultures are added and the product is fermented for about 16 hours at 40° C. Finally, the yogurt alternative is heated to 85° C. before cooling thereof to 30° C. and filling thereof is carried out.

### Application Example

Fermented and unfermented proteins were each used to produce a vegan cheese, and the sensory properties were then assessed in each case on a scale of (1)=weak to (5)=strong. The results are reported in Table 1. Examples 1 and 2 are in accordance with the invention, and examples V1 and V2 serve for comparison.

TABLE 1
Composition of vegan cheeses—amounts in % by weight

| Component | V1 | 1 | V2 | 2 |
|---|---|---|---|---|
| Solid coconut oil | 20.0 | 20.0 | 20.0 | 20.0 |
| Shea butter | 4.0 | 4.0 | 4.0 | 4.0 |
| Oat protein (unfermented) | 5.0 | — | — | — |
| Oat protein, example 1 | — | 5.0 | — | |
| Lupin protein (unfermented) | — | — | 5.0 | — |
| Lupin protein, example 2 | — | — | — | 5.0 |
| Salt | 1.5 | 1.5 | 1.5 | 1.5 |
| Carrageenan | 0.5 | 0.5 | 0.5 | 0.5 |
| Starch | 24.5 | 24.5 | 24.5 | 24.5 |
| Dextrose | 0.5% | 0.5% | 0.5% | 0.5% |
| Flavourings | 0.5 | 0.5 | 0.5 | 0.5 |
| Water | to 100% by weight | | | |
| Properties of the proteins | | | | |
| Colour brightness | 3 | 5 | 3 | 5 |
| Bitter taste | 4 | 1 | 5 | 2 |
| Acidic odour note | 1 | 3 | 1 | 3 |
| Properties of the end product | | | | |
| Creaminess | 2 | 5 | 2 | 5 |
| Mouthfeel | 2 | 5 | 2 | 5 |
| Stickiness | 2 | 1 | 3 | 1 |

Compared to the unfermented products, the fermented plant proteins exhibit a brighter colour, practically no bitter taste and a pleasant acidic odour note. The end products according to the invention are creamier, are less sticky and have a higher mouthfeel than the comparative products.

1. Fermented plant protein obtained by the following steps
(a) providing a plant protein source;
(b) admixing the protein from step (a) with water to obtain an aqueous protein phase;
(c) admixing the aqueous protein phase from step (b) with microorganisms suitable for fermentation of proteins and/or with proteases; and
(d) fermenting the aqueous protein phase from step (c); and optionally (e) inactivating and removing the microorganisms and/or enzymes.

2. Method for producing fermented plant protein, comprising the following steps:
(a) providing a plant protein source;
(b) admixing the protein from step (a) with water to obtain an aqueous protein phase;
(c) admixing the aqueous protein phase from step (b) with microorganisms suitable for fermentation of proteins and/or with proteases; and
(d) fermenting the aqueous protein phase from step (c); and optionally
(e) inactivating and removing the microorganisms and/or enzymes.

3. Method according to claim 2, comprising using plant protein selected from the group consisting of by broad bean proteins, oat proteins, rice proteins, almond proteins, acorn proteins, potato proteins, soya proteins, pea proteins, lupin proteins, rapeseed proteins, sunflower proteins, and pumpkin seed proteins, and mixtures thereof.

4. Method according to claim 2, comprising using plant protein which has have been previously subjected to a pressure/temperature pretreatment.

5. Method according to claim 2, comprising adding at least one plant fat and/or at least one dietary fibre to the aqueous protein phase from step (b).

6. Method according to claim 5, comprising using plant fat selected from the group consisting of cocoa butter, coconut oil, palm oil, palm kernel oil, solid coconut oil, nut oil, shea butter, apricot oil, soya lecithin, sunflower oil, soya oil, rapeseed oil, nut oil, and mixtures thereof.

7. Method according to claim 5, comprising using plant fibre as dietary fibre.

8. Method according to claim 2, comprising using microorganisms selected from the group of mesophilic and thermophilic microorganisms and acidifiers formed by metabolism with or without the use of a substrate.

9. Method according to claim 2, comprising using enzymes selected from the group consisting of pepsin, chymosin, cathepsin E, papain, cathepsin K, caspase, calpain, chymotrypsin, plasmin, thrombin, trypsin, granzymes, kallikrein, proteasome, and mixtures thereof.

10. Method according to claim 2, comprising carrying out the fermentation at a temperature in the range from about 30° C. to about 55° C. and/or a pH in the range from about 3.5 to about 7.0.

11. Method according to claim 2, comprising following the fermentation with a purification and/or concentration step.

12. Method according to claim 2, comprising carrying out the fermentation in multiple stages.

13. Method according to claim 2, comprising producing a vegan dairy substitute.

14. Vegan dairy substitute containing the fermented plant protein according to claim 1.

15. Method for producing a vegan dairy substitute, comprising the following steps:
(i) providing a vegan dairy substitute base;
(ii) providing the fermented plant protein according to claim 1; and
(iii) mixing components (i) and (ii).
